(19) United States
(12) Patent Application Publication
(10) Pub. No.: US 2023/0146808 A1
(43) Pub. Date: May 11, 2023

(54) INJECTION DEVICE STORAGE CONTAINER

(71) Applicant: Sanofi, Paris (FR)

(72) Inventors: Uwe Dasbach, Frankfurt am Main (DE); Kai Scheinert, Frankfurt am Main (DE); Daniel Auernhammer, Frankfurt am Main (DE); Florian Schauderna, Frankfurt am Main (DE); Thomas Mark Kemp, Herts Cambridgeshire (GB); Tim Schuller, Herts Cambridgeshire (GB); Robbie Wilson, Herts Cambridgeshire (GB); Michael Noble, Herts Cambridgeshire (GB); Ryan Anthony McGinley, Herts Cambridgeshire (GB)

(21) Appl. No.: 17/915,344

(22) PCT Filed: Apr. 1, 2021
(86) PCT No.: PCT/EP2021/058753

§ 371 Date: Sep. 28, 2022

(30) Foreign Application Priority Data
Apr. 3, 2020 (EP) 20315114.7

(51) Int. Cl. A61M 5/00; A61M 5/44; F25D 25/00

(52) U.S. Cl. CPC A61M 5/008; A61M 5/44; A61M 5/002; F25D 25/005; A61M 2205/3606

(57) ABSTRACT

An injection device storage container includes a case configured to contain at least one injection device for delivering a medicament; and a hanger arrangement coupled to the case and configured to suspend the case from a supporting wall; wherein the case includes an upper panel having an opening formed therein through which the at least one injection device can be dispensed, and wherein the opening is arranged such that, when the case is suspended from the supporting wall, the at least one injection device is dispensed in a horizontal orientation through the opening.

### CROSS REFERENCE TO RELATED APPLICATIONS

The present application is the national stage entry of International Patent Application No. PCT/EP2021/058753, filed on Apr. 1, 2021, and claims priority to Application No. EP 20315114.7, filed on Apr. 3, 2020, the disclosures of which are incorporated herein by reference.

### TECHNICAL FIELD

The present disclosure relates to an injection device storage container for storing one or more injection devices, for example in a refrigerator.

### BACKGROUND

Patients suffering chronic disease require regular treatment with medicaments, e.g., on the basis of a predefined schedule. Particular medicaments require refrigerated storage, and are often stored refrigerated in a household refrigerator, also known as a fridge. In a home treatment environment, the patient stores the medicament in their own refrigerator and administers a predefined dose as required. Hence, the medicament is typically provided in a secondary packaging for convenient placement and storage in the household refrigerator. However, the medicament must be stored together with other items that require constant refrigeration, such as foodstuffs and beverages.

Depending on the dosage form of the medicament, the secondary packaging containing the medicament may store a primary packed medicament itself, or may store one or more different kinds of drug delivery devices. For instance, the medicament may be provided in a pre-filled syringe or pen-type injector.

There is a desire to improve the storage of injection devices, for example in a refrigerator.

### SUMMARY

It is an aspect of the present disclosure to provide an injection device storage container for storing a plurality of injection devices.

According to the present disclosure, there is provided an injection device storage container comprising a case configured to contain at least one injection device for delivering a medicament; and a hanger arrangement coupled to the case and configured to suspend the case from a supporting wall; wherein the case comprises an upper panel having an opening formed therein through which the at least one injection device can be dispensed, and wherein the opening is arranged such that, when the case is suspended from the supporting wall, the at least one injection device is dispensed in a horizontal orientation through the opening.

The opening may be dimensioned so that only one injection device of the at least one injection devices can be dispensed through the opening at a time.

The injection device storage container may further comprise a guiding arrangement located within the case, wherein the guiding arrangement defines a path along which the at least one injection device can be guided towards the opening.

The guiding arrangement may comprise a conduit through which the at least one injection device can move.

The guiding arrangement may comprise a sloping lower guide surface, wherein the lower guide surface slopes upwards towards the opening in the upper panel of the case.

The injection device storage container may further comprising a propulsion mechanism configured to propel the at least one injection device contained in the case towards the opening.

The propulsion mechanism may comprise a resilient member configured to apply a force to the at least one injection device to move it towards the opening.

The injection device storage container may further comprise a restraining member arranged adjacent the opening to inhibit removal of the at least one injection device from the opening.

The hanger arrangement may comprise a hook configured to hook over a top edge of the supporting wall.

The injection device storage container may further comprise a display located on the hook, the display configured to display information to a user indicative of a condition of the injection device storage container or a condition of one or more injection devices contained within the case.

The display may be configured to display at least one of a temperature of the injection device storage container, a number of injection devices contained within the case, or an indication of a length of time until an injection is due.

The injection device storage container may further comprise a cold pack arranged within the case to moderate an internal temperature of the case.

The hanger arrangement may be integrally formed with the case.

According to the present disclosure, there is also provided a system comprising an injection device storage container as disclosed herein and at least one injection device contained within the injection device storage container.

The at least one injection device may comprise a container containing a medicament.

### BRIEF DESCRIPTION OF THE FIGURES

Embodiments of the invention will now be described, by way of example only, with reference to the accompanying drawings, in which:

FIG. 1A is a schematic side view of an injection device suitable for storage in the injection device storage container of the present disclosure;

FIG. 1B is a schematic side view of the injection device of FIG. 1A, with the cap removed from the housing;

FIG. 2 is an isometric view of an injection device storage container according to embodiments of the present disclosure;

FIG. 3 is an isometric view of the injection device storage container of FIG. 2 when attached to the wall of a refrigerator door shelf;

FIG. 4 is an isometric view of the a refrigerator having the injection device storage container of FIG. 2 attached to the wall of a refrigerator door shelf;

FIG. 5 is a schematic side view of the injection device storage container of FIG. 2.

### DETAILED DESCRIPTION

The present disclosure provides an injection device storage container configured to contain and store at least one injection device for delivering a medicament. An injection device is an example of a drug delivery device and may be a pen-injector, an auto-injector or the like. The injection device storage container may be stored in a household refrigerator, also known as a fridge.

The patient may receive the injection device storage container in an empty condition. When the patient is supplied with one or more injection devices they can be loaded into the injection device storage container. The injection device storage container may be placed in the refrigerator until a scheduled dosing time is due.

The injection device storage container may include a hanger arrangement to allow a patient to locate the injection device storage container in various positions in their refrigerator. For example, the injection device storage container may be located for convenient access to make it easier for a patient to be reminded that an injection is due. Depending on the patient's needs, the storage container may be positioned discreetly in the refrigerator (e.g., to keep the storage container from children), or positioned prominently within the refrigerator to serve as a constant reminder when they open their refrigerator door. The injection device storage container may be conveniently positioned out of the way of food in the refrigerator, in an accessible position. The storage container may also be moved if the patient desires, or temporarily removed for periodic cleaning. Various storage container embodiments are described below.

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 3 ml. Another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml). Yet another device may comprise a pre-filled syringe within a housing of the device. The syringe may be fixed within the housing or may be moveable within the housing, for example from a retracted position to an operation extended position.

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 17 and 29 Gauge.

The delivery devices described herein can also include one or more automated functions. For example, one or more of combining the needle and cartridge, needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include an actuator, for example, one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of combining the needle and cartridge, needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

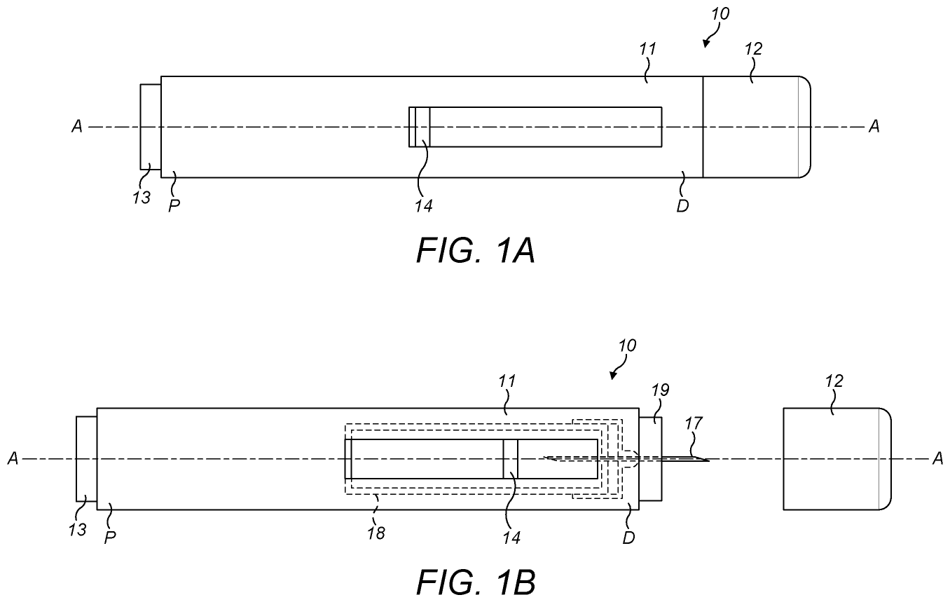

An exemplary drug delivery device 10, also known as an injection device, suitable for being stored in the injection device storage container of the present disclosure is shown in FIGS. 1A and 1B. Device 10, as described above, is configured to inject a medicament into a patient's body. Device 10 includes a housing 11 which typically contains a cartridge or pre-filled syringe that defines a reservoir containing the medicament to be injected, and the components required to facilitate one or more steps of the delivery process.

The device 10 can also include a cap 12 that can be detachably mounted to the housing 11. Typically, a user must remove cap 12 from housing 11 before device 10 can be operated.

As shown, housing 11 is substantially cylindrical and has a substantially constant diameter along the longitudinal axis A-A. The housing 11 has a distal region D and a proximal region P. The term “distal” refers to a location that is relatively closer to a site of injection, and the term “proximal” refers to a location that is relatively further away from the injection site.

Device 10 can also include a needle sleeve 19 coupled to housing 11 to permit movement of sleeve 19 relative to housing 11. For example, sleeve 19 can move in a longitudinal direction parallel to longitudinal axis A-A. Specifically, movement of sleeve 19 in a proximal direction can permit a needle 17 to extend from distal region D of housing 11.

Insertion of needle 17 can occur via several mechanisms. For example, needle 17 may be fixedly located relative to housing 11 and initially be located within an extended needle sleeve 19. Proximal movement of sleeve 19 by placing a distal end of sleeve 19 against a patient's body and moving housing 11 in a distal direction will uncover the distal end of needle 17. Such relative movement allows the distal end of needle 17 to extend into the patient's body. Such insertion is termed “manual” insertion as needle 17 is manually inserted via the patient's manual movement of housing 11 relative to sleeve 19.

Another form of insertion is “automated”, whereby needle 17 moves relative to housing 11. Such insertion can be triggered by movement of sleeve 19 or by another form of activation, such as, for example, a button 13. As shown in FIGS. 1A and 1B, button 13 is located at a proximal end of housing 11. However, in other embodiments, button 13 could be located on a side of housing 11.

Other manual or automated features can include drug injection or needle retraction, or both. Injection is the process by which a bung or piston 14 is moved from a proximal location to a more distal location within the reservoir of the medicament container 18 in order to force a medicament from the container 18 through needle 17. In some embodiments, a drive spring (not shown) is under compression before device 10 is activated. A proximal end of the drive spring can be fixed within proximal region P of housing 11, and a distal end of the drive spring can be configured to apply a compressive force to a proximal surface of piston 14. Following activation, at least part of the energy stored in the drive spring can be applied to the proximal surface of piston 14. This compressive force can act on piston 14 to move it in a distal direction. Such distal movement acts to compress the liquid medicament within the container 18, forcing it out of needle 17.

Following injection, needle 17 can be retracted within sleeve 19 or housing 11. Retraction can occur when sleeve 19 moves distally as a user removes device 10 from a patient's body. This can occur as needle 17 remains fixedly located relative to housing 11. Once a distal end of sleeve 19 has moved past a distal end of needle 17, and needle 17 is covered, sleeve 19 can be locked. Such locking can include locking any proximal movement of sleeve 19 relative to housing 11.

Another form of needle retraction can occur if needle 17 is moved relative to housing 11. Such movement can occur if the cartridge 18 within housing 11 is moved in a proximal direction relative to housing 11. This proximal movement can be achieved by using a retraction spring (not shown), located in distal region D. A compressed retraction spring, when activated, can supply sufficient force to the cartridge 18 to move it in a proximal direction. Following sufficient retraction, any relative movement between needle 17 and housing 11 can be locked with a locking mechanism. In addition, button 13 or other components of device 10 can be locked as required.

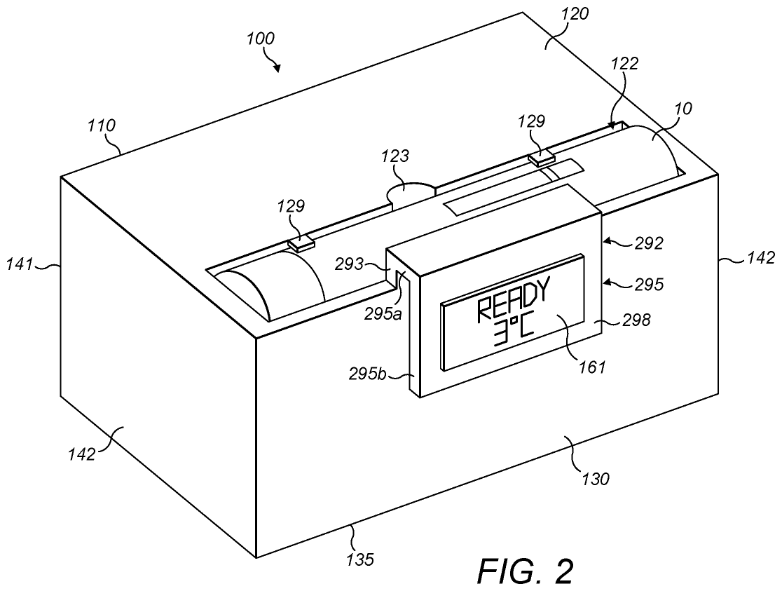

With reference to FIGS. 2 to 5, an injection device storage container 100 according to exemplary embodiments is shown. The injection device storage container 100 is configured to store a plurality of injection devices such as the injection device 10 previously described in relation to FIGS. 1A and 1B.

FIG. 2 shows the injection device storage container 100 comprising a case 110. The case 110 comprises a front wall 130, a rear wall 141, and two side walls 142. The case 110 also has a base 135 and an upper panel 120, also known as a lid. The upper panel 120 may be removable, or may be permanently attached to the walls 130, 141, 142. The upper panel 120 forms the top of the case 110.

The case 110 is configured to hold and store a plurality of injection devices 10. In particular, the case 110 is configured to contain a plurality of injection devices 10 such as the injection devices 10 of FIGS. 1A and 1B in a horizontal orientation. This means that the injection devices 10 are orientated such that the longitudinal axis of each injection device 10 is substantially parallel to the base 135 and the upper panel 120 of the case 110, when the injection device storage container 100 is suspended from a refrigerator shelf wall as discussed later.

A width of the case 110, measured between the two side walls 142, is sufficient to accommodate the length of an injection device 10 such as the injection device 10 previously described in relation to FIGS. 1A and 1B. The width of the case may be between about 150 mm and about 250 mm. A height of the case 110, measured between the base 135 and the upper panel 120, is sufficient to accommodate the width of at least one injection device 10. The height of the case may be between about 30 mm and about 200 mm. A depth of the case 110, measured between the front wall 131 and the rear wall 141, is sufficient to accommodate the width of at least one injection device 10. The depth of the case may be between about 30 mm and about 180 mm.

As shown in FIG. 2, the upper panel 120 extends from a top edge of the front wall 131 to a top edge of the rear wall 141. An opening 122 is located in the upper panel 120 of the case 110, through which the injection devices 10 stored inside the case 110 may be removed or otherwise dispensed. The injection devices 10 are therefore removed through the top of the case 110, in a direction substantially away from the ground when the injection device storage container 100 is suspended in a refrigerator. The opening 122 is arranged so that the injection devices 10 are dispensed outside the case 110 in their horizontal orientation—in other words sideways, or tangential to the upper panel 120—through the opening 122.

An injection device 10 may be dispensed through the opening 122 by a user physically grasping the injection device 10 nearest the opening 122 and removing the injection device 10 from the container 100. In other examples, the injection device 10 may be automatically dispensed by the injection device storage container 100, for example by an electromechanical dispensing mechanism.

The opening 122 may have a rectangular shape. A width of the opening 122 is sufficient to accommodate the width of an injection device 10, while a length of the opening 122 is sufficient to accommodate the length of an injection device 10. The opening 122 may be dimensioned so that only one injection device 10 may be removed at a time. This may assist with patient compliance of an injection regimen.

The opening 122 may have a recess portion 123 formed in the upper panel 120 to assist a user in removing an injection device 10 from the case 110. The recess portion 123 may be dimensioned to receive at least part of a user's finger to assist the user in grasping the injection device 10 for removal from the case 110. More than one recess portion 123 may be formed in the opening 122. For example there may be two recess portions 123, with one recess portion 123 formed on each opposing side of the opening 122 so that a user may place a finger in each recess portion 123, either side of the injection device 10.

The case 110 may optionally have one or more restraining members 129 arranged adjacent the opening 122 for inhibiting removal of an injection device 10 from the injection device storage container 100. The restraining member 129 inhibits removal of an injection device 10 by making it more difficult to remove the injection device 10 than if the restraining member 129 were not present.

FIG. 2 shows two restraining members 129, however there may be more restraining members 129 than this, or fewer. The one or more restraining members 129 are formed as one or more protrusions extending from the upper panel 120, partially across the opening 122, to impede removal of an injection device 10 from the opening 122. The restraining member 129 may protrude between 1 mm and 5 mm into the opening 122 from an edge of the opening 122, for example.

The restraining member 129 may be formed of a resilient or flexible material which allows the restraining member 129 to be deformed upon application of a force on the restraining member 129. The restraining member 129 may prevent an injection device 10 from being removed from the opening 122 of the case 110 unless a removal force greater than a threshold value is applied to the restraining member 129 via the injection device 10.

The front wall 131, the rear wall 141, the two side walls 142, the base 135 and the upper panel 120 may be formed from an opaque material, for example, an opaque plastic material. In other examples, one or more of the front wall 131, the rear wall 141, the two side walls 142, the base 135 and the upper panel 120 may wholly or in part be formed from a transparent material, or formed from a translucent or frosted material, for example, a clear plastic material with a frosted coating or a treated surface. A portion of the upper panel 120 may be transparent so that a user may view the interior of the case 110 to identify the number of injection devices 10 contained within the case 110.

The injection device storage container 100 further comprises a hanger arrangement 292. The hanger arrangement 292 is coupled to the case 110 and is configured to hang the injection device storage container 100 from a supporting object such as a wall of a refrigerator shelf.

The hanger arrangement 292 comprises an extending part 293 and a hook 295. The extending part 293 is arranged to extend away from the case 110, in particular away from the upper panel 120 of the case 110. The hook 295 is arranged at an end of the extending part 293 furthest from the case 110. The hook 295 is configured to fit over a supporting body. The case 110 hangs directly below the hook 295 at a lower end of the extending part 293.

The extending part 293 may extend from the case 110 substantially in the same plane as the front wall 130. When the hanger arrangement 292 is hanging from a supporting object, the extending part 293 extends upwards.

The extending part 293 is a rectangular shape. The length of the extending part 293, which is measured along the top edge of the front wall 130, may be between about 50 mm and about 200 mm. The height of the extending part 293 may be between about 1 mm and about 50 mm.

The extending part 293 of the hanger arrangement 292 may be attached to the case 110 by welding or bonding, or another suitable means of coupling. Alternatively, the hanger arrangement 292 and case 110 may be formed from a single piece, such as by casting or machining. For example, the hanger arrangement 292 and at least one of the upper panel 120 and the front wall 130 may be integrally formed, such as through an injection moulding process.

The hook 295 extends from an end of the extending part 293 which is furthest from the case 110. The hook 295 is configured to engage with a supporting object such as a wall of a refrigerator door shelf. The hook 295 is shaped to fit over the supporting object such that the extending part 293 of the hanger arrangement 292 hangs beneath a top edge of the supporting object.

With respect to the extending part 293, the hook 295 extends out of the plane of the extending part 293 in a direction away from the front wall 130. That is, the hook 295 is disposed away from the case 110. The hanger arrangement 292 is configured to support the case 110 directly below the hook 295, when the hook 295 is hanging from a supporting object.

The hook 295 comprises a first part 295a and a second part 295b. The first part 295a extends from the end of the extending part 293. The first part 295a extends perpendicular to the extending part 293, in a forward direction from the extending part 293. That is, the first part 295a extends in a direction away from the case 110, in a direction perpendicular to the plane of the front wall 130.

The second part 295b extends from an end of the first part 295a which is furthest from the extending part 293. The second part 295b extends perpendicular to the first part 295a. The second part 295b extends in a sideways direction from the first part 295a. That is, the second part 295b extends along an axis which is parallel to the extending part 293, and substantially parallel to the front wall 130.

The length of the first part 295a, when measured in a direction away from the front wall 130 of the case 110, may be between about 5 mm and about 20 mm. The length of the second part 295b, when measured extending from the first part 295b may be between about 15 mm and about 50 mm. The hanger arrangement 292 may be deflected to form the hook 295. The hook 295 may be formed having square corners where deflected, as shown in FIG. 2. Alternatively, the hook 295 may be rounded. The hanger arrangement 292 may be bent or folded to form the hook 295. Alternatively, a second piece of material may be attached to the extending part 293 at a right angle to form the first part 295a, and a third piece of material may be attached to the first part 295a to form the second park 295b.

In some examples, the extending part 293 may not be present. For example, the hook 295 may be coupled to the case 110 without an extending part 293 between the case 110 and the hook 295. In such an example, the first part 295a of the hook 295 may be coupled to, and extend from, the front wall 130. The first part 295a extends in a forward direction from the front wall 130. That is, the first part 295a extends in a direction away from the case 110.

The hanger arrangement 292 allows the case 110 of the injection device storage container 100 to be suspended inside a household refrigerator. In particular, the hanger arrangement 292 can be hung from a door shelf of the refrigerator.

The injection device storage container 100 may also comprise a display 161. The display 161 may be any form of display 161 suitable for conveying information to a patient, for example an electronic display such as an LCD display or one or more LEDs. The display 161 may be configured to display information to a user related to a condition of the injection device storage container 100 and/or a condition of one or more injection devices 10 contained within the case 110. For example, the display may be configured to display information representing a temperature of the injection device storage container 100 or a number of injection devices 10 contained within the case 110. In other examples the information may represent a time, for example an indication of a length of time until an injection is due, or a length of time since an injection device was removed from the case 110.

The display 161 is arranged on the hook 190, in particular on a front surface 298 of the second part 295b of the hook. The front surface 290 is on the opposite side of the hook 190 to the extending part 293 and case 110, and faces away from the front wall 130 of the case. Positioning the display on the front surface 298 of the hook 190 allows a patient to easily view displayed information regarding a condition of the injection device storage container 100 and/or injection devices 10 contained within.

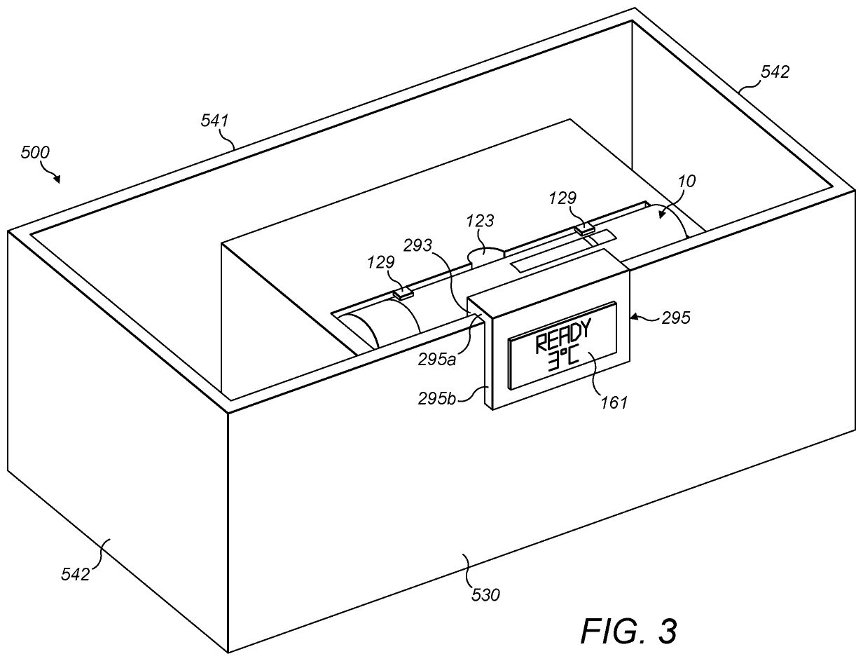

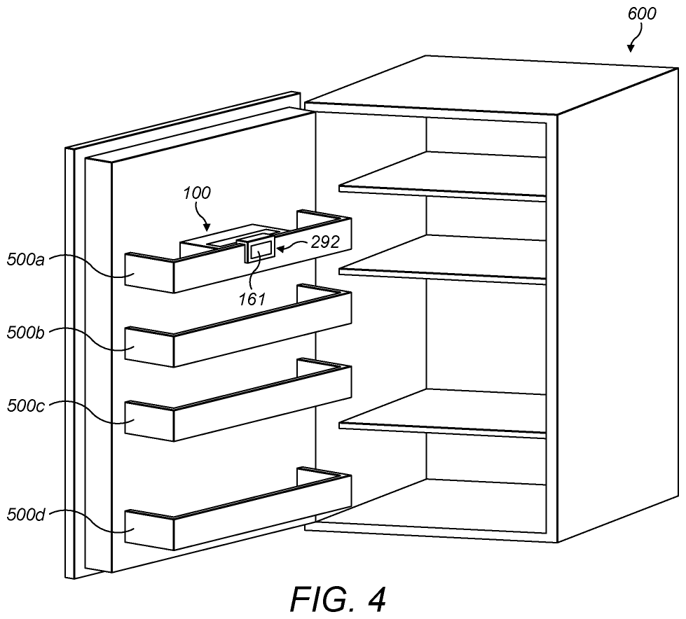

FIGS. 3 and 4 show the injection device storage container 100 of FIG. 2 when located in a typical refrigerator door shelf 500 of a refrigerator 600. A typical refrigerator door shelf 500 is a tray shape having a base and four vertical walls, namely a front wall 530, a rear wall 541 and a pair of side walls 542. The tray is mounted on horizontal rails formed in the internal face of the refrigerator door and can be slidably removed in a direction perpendicular to the internal face of the refrigerator door.

The hook 295 can be placed over the front wall 530 of the tray which faces away from the internal face of the refrigerator door when the tray is mounted thereon. The second part 295b of the hook is located adjacent the front surface of the front wall 530 of the refrigerator door shelf 500, while the front wall 130 of the case 110 is located adjacent the rear surface of the front wall 530 of the refrigerator door shelf 500. The injection device storage container 100 is suspended within the refrigerator door shelf 500, in the space defined by the four vertical walls and base.

By providing a hanger arrangement 292 for suspending the injection device storage container 100 within the refrigerator door shelf 500, the injection device storage container 100 can be placed in a prominent position in the refrigerator door shelf 500 while still being isolated from foodstuffs. Suspending the injection device storage container 100 may allow other items to be stored in the refrigerator 600 in a space created underneath the base 135 of the storage container 100, thereby better utilising the limited space of the refrigerator 600.

The injection device storage container 100 can be located in a prominent position on the refrigerator door. The injection device storage container 100 is located away from food and the like in the refrigerator. The injection device storage container 100 is unlikely to be covered or knocked in this location.

When the injection device storage container 100 is hung from the front wall 530 of the refrigerator door shelf 500 using the hanger arrangement 292, the front wall 130, rear wall 141, and two side walls 142 of the injection device storage container 100 are substantially parallel to the walls of the refrigerator door shelf 500. The base 135 and the upper panel 120 of the case 110 are substantially parallel to the base of the refrigerator door shelf 500. The front wall 130 faces the patient when the refrigerator door is opened. The patient can see the upper panel 120 of the case 110 and the opening 122 when viewing the refrigerator door shelf 500 from above.

As can be seen in FIG. 3, the injection device storage container 100 is substantially contained within the refrigerator door shelf, reducing visibility of the injection device storage container 100 and ensuring the injection device storage container is out of the way of the inside of the refrigerator. However the presence of the hanger arrangement 292 at the front wall of the refrigerator door shelf allows for simple attachment and detachment of the injection device storage container from the refrigerator door shelf. The hook 190 of the hanger arrangement 292 being visible from the front of the refrigerator door shelf front wall allows a patiently to quickly and easily identify the location of the injection device storage container 100 within the refrigerator, which may be particularly useful where the refrigerator door shelves 500a, 500b, 500c, 500d are opaque, or where the injection device storage container 100 is located in one of the upper refrigerator door shelves 500a that would be difficult for a patient to otherwise see the contents of.

The location of the display 161 on the hook 295 means that it is clearly visible from outside the refrigerator door shelf, allowing a patient to easily be presented with information regarding a condition of the injection device storage container and/or injection devices 10 it contains. The user may therefore be able to suspend the injection device storage container 100 from the uppermost refrigerator door shelf 500a of the refrigerator 600 and still view the displayed information.

It can be seen in FIG. 3 that the location of the opening 122 at the upper panel 120 of the case 110 allows a patient to have easy access to the injection devices 100 contained in the injection device storage container 100. The injection devices 10 are arranged horizontally in the injection device storage container 100 so that the longitudinal axis of each injection device 10 is parallel to the plane of the front wall 130. Storing and dispensing the injection devices 10 in a horizontal orientation allows for easy retrieval of an injection device 10 by a patient, for example those patients with reduced dexterity.

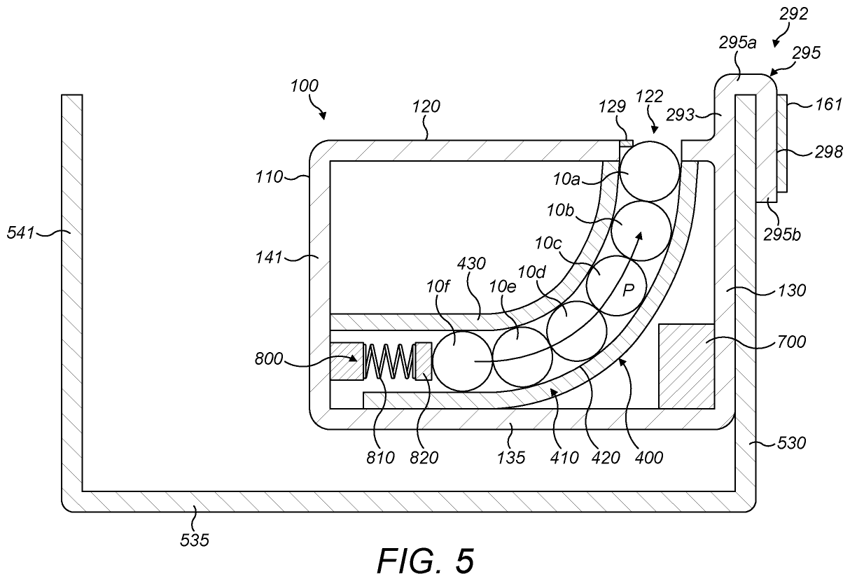

FIG. 5 shows a schematic cross section of the injection device storage container 100 of FIG. 3 when viewed from the side.

An injection device guiding arrangement 400 is located within the case 110. The guiding arrangement 400 is configured to guide the injection devices 10a-f contained within the case 110 towards the opening 122, so that they can be dispensed. The guiding arrangement 400 defines a path P along which the plurality of injection devices 10a-f can be guided towards the opening 122.

The guiding arrangement 400 is arranged to contain each of the injection devices 10 in the horizontal orientation described previously. As can be seen in FIG. 5, the injection devices 10a-f are arranged horizontally by the guiding arrangement 400 such that the longitudinal axis of each injection device 10a-f is substantially parallel to the plane of the upper panel 120, and to the front wall 130. The longitudinal axis of each injection device 10a-f shown in FIG. 5 extends perpendicular to the page.

The guiding arrangement 400 may be formed as a conduit through which the injection devices 10a-f may move towards the opening 122, while remaining in the horizontal orientation. For example, the injection devices 10a-f could slide towards the opening 122 or roll towards the opening 122.

FIG. 5 shows the guiding arrangement 400 comprising a sloping lower guide surface 420, wherein the lower guide surface 420 slopes upwards towards the opening 122 in the upper panel 120 of the case 110. An injection device 10a-f moving along the path P formed by the guiding arrangement 400 moves up the sloping lower guide surface 420 towards the opening 122. The lower guide surface 420 may comprise a ramp, or may comprise one or more rails along which the injection devices 10 may slide or roll.

The guiding arrangement 400 may also comprise an upper guide surface 430 arranged above the lower guide surface 420, that is nearer the upper panel 120 of the case 110. The upper guide surface 430 and lower guide surface 420 are separated by a predefined distance in order to form a passage 410 through which the injection devices 10a-f can be guided along the path P.

The distance between the upper guide surface 430 and lower guide surface 420 may be substantially constant along the path P formed between upper guide surface 430 and lower guide surface 420 so that the passage has a constant height. The distance between the upper guide surface 430 and lower guide surface 420 is at least large enough to accommodate the width of an injection device 10. The distance between the upper guide surface 430 and lower guide surface 420 may substantially correspond to the width of a single injection device 10 so that the injection devices 10a-f are tightly constrained and guided by the upper guide surface 430 and lower guide surface 420.

The injection device storage container 100 may have a propulsion mechanism 800 located within the case 110. The propulsion mechanism 800 is arranged to propel the injection devices 10 towards the opening 122 so that they can be dispensed. As discussed previously, the opening 122 through which the injection devices 10 are dispensed is formed in the upper panel 120 of the case 110. The propulsion mechanism 800 can therefore assist in transporting injection devices 10a-f from inside the lower part of the case 110 towards the upper part of the case 11, along the path P formed by the guiding arrangement 400, so that they can be dispensed from the top of the case 110.

FIG. 5 shows the propulsion mechanism 400 comprising a resilient member 810 such as a spring. One end of the resilient member 810 is coupled to the case 110, for example the rear wall 141 of the case 110. The other end of the resilient member 810 is coupled to a contact member 820. When one or more injection devices 10a-f are contained within the case 110, the contact member 820 is arranged to be in contact with at least one of the injection devices 10a-f. FIG. 5 shows the contact member 820 in contact with the rearmost injection device 10f.

In some examples, no contact member 820 is present and the resilient member 810 contacts one or more of the injection devices 10 directly.

The resilient member 810 exerts a force on the injection device 10f via the contact member 820, urging the injection device 10f towards the opening 122 so that it can be dispensed. Where the case 110 contains a plurality of injection devices 10a-f, as shown in FIG. 5, the force applied by the resilient member 810 may be applied to all of the injection devices 10a-f, causing them each to be biased towards the opening 122.

Alternatively, or additionally, other suitable forms of propulsion mechanism 400 may be used such as an electromechanical assembly.

FIG. 5 shows a restraining member 129 located adjacent the opening 122 of the case 110. The restraining member 129 may counterbalance the force applied to the injection devices 10a-f by the propulsion mechanism 800. The restraining member 129 may therefore inhibit the injection devices 10 from being fully dispensed out of the opening 122 by the propulsion mechanism 800, without patient intervention.

FIG. 5 also shows the injection device storage container 100 comprising a cold pack 700. The cold pack 700 may alternatively be known as an ice pack, however it should be noted that the term ice pack does not necessarily mean that the ice pack is at a temperature below the freezing point of water. The cold pack 700 is located within the case 110. The cold pack 700 moderates the temperature of the injection device storage container 100 and the injection devices 10a-f held inside. The cold pack 700 may comprise a phase change material (PCM).

The cold pack 700 is formed from a material that is slow to change temperature. When the injection device storage container 100 is located inside a closed refrigerator 600, the cold pack 700 reaches an equilibrium temperature with the internal temperature of the refrigerator 600. When the refrigerator door is opened, the immediate environment around the injection device storage container 100 may begin to increase in temperature due to exposure to warmer air outside the refrigerator 600. However, the cold pack 700 within the case 110 resists the increase in temperature, moderating the temperature of the inside of the case 110 and the injection devices 10a-f held within, keeping them cold. The cold pack 700 may be of particular use where the injection device storage container 100 is completely removed from the refrigerator 600, for example when the injection device storage container 100 is portable and is being transported by a patient.

One or more of the front wall 131, the rear wall 141, the two side walls 142, the base 135 and the upper panel 120 of the case 100 may be made from a thermally insulating material. This can help shield the injection devices 10 contained within the injection device storage container 100 from temperature variations outside the case 110. The thermally insulating material may keep the injection devices 10 colder for a longer period of time compared to use of a non-thermally insulting material.

Relative orientations disclosed herein are to be interpreted from the viewpoint of when the injection device storage container 100 is suspended from a supporting wall of a refrigerator using the hanger arrangement 292, as shown in FIGS. 3, 4 and 5. For example, when the injection device storage container 100 is suspended from the front wall 530 of a refrigerator door shelf 500 using then hanger arrangement 292, the front wall 130 of the case 110 is the wall nearest the front wall 530 of the refrigerator door shelf 530, the rear wall 141 of the case 110 is the wall furthest the front wall 530 of the refrigerator door shelf 530, the base 135 of the injection device storage container 100 forms the surface nearest the base of the refrigerator door shelf 500, and the upper panel 120 forms the upper surface of the case 110, opposite and above the base 135 relative to the ground.

With respect to the injection devices 10 described herein, the term ‘horizontal orientation’ means an orientation in which the longitudinal axis of the injection device 10 is parallel to the ground, and parallel to the base 135 of the case 110 and refrigerator door shelf 500 when the injection device storage container 100 is suspended from the front wall 530 of the refrigerator door shelf 500.

The injector devices described herein are configured to receive either a cartridge of medicament or a syringe pre-filled with a medicament. Herein, the term “medicament container” is intended to encompass both a cartridge of medicament and a pre-filled syringe.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injector device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injector devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(ω-carboxyheptadecanoyl)-des(B30) human insulin and B29-N-(ω-carboxyhepta¬decanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993 (a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

1-15. (canceled)

16. An injection device storage container for storing in a household refrigerator, the injection device storage container comprising:
a case configured to contain at least one injection device for delivering a medicament; and
a hanger arrangement coupled to the case and configured to suspend the case from a supporting wall of the refrigerator;
wherein the case comprises an upper panel having an opening formed therein through which the at least one injection device can be dispensed, and
wherein the opening is arranged such that, when the case is suspended from the supporting wall, the at least one injection device is dispensed in a horizontal orientation through the opening.

17. The injection device storage container according to claim 16, wherein the opening is dimensioned so that only one injection device of the at least one injection device can be dispensed through the opening at a time.

18. The injection device storage container according to claim 16, further comprising a guiding arrangement located within the case, wherein the guiding arrangement defines a path along which the at least one injection device can be guided towards the opening.

19. The injection device storage container according to claim 18, wherein the guiding arrangement comprises a conduit through which the at least one injection device can move.

20. The injection device storage container according to claim 18, wherein the guiding arrangement comprises a sloping lower guide surface, wherein the lower guide surface slopes upwards towards the opening in the upper panel of the case.

21. The injection device storage container according to claim 16, further comprising a propulsion mechanism configured to propel the at least one injection device contained in the case towards the opening.

22. The injection device storage container according to claim 21, wherein the propulsion mechanism comprises a resilient member configured to apply a force to the at least one injection device to move it towards the opening.

23. The injection device storage container according to claim 16, further comprising a restraining member arranged adjacent the opening to inhibit removal of the at least one injection device from the opening.

24. The injection device storage container according to claim 16, wherein the hanger arrangement comprises a hook configured to hook over a top edge of the supporting wall.

25. The injection device storage container according to claim 24, further comprising a display located on the hook, the display configured to display information to a user indicative of a condition of the injection device storage container.

26. The injection device storage container according to claim 25, wherein the display is configured to display at least one of a temperature of the injection device storage container, a number of injection devices contained within the case, or an indication of a length of time until an injection is due.

27. The injection device storage container according to claim 24, further comprising a display located on the hook, the display configured to display information to a user indicative of a condition of the at least one injection device contained within the case.

28. The injection device storage container according to claim 16, further comprising a cold pack arranged within the case to moderate an internal temperature of the case.

29. The injection device storage container according to claim 16, wherein the hanger arrangement is integrally formed with the case.

30. A system comprising:
an injection device storage container comprising:
a case containing at least one injection device for delivering a medicament; and
a hanger arrangement coupled to the case and configured to suspend the case from a supporting wall of the refrigerator;
wherein the case comprises an upper panel having an opening formed therein through which the at least one injection device can be dispensed, and wherein the opening is arranged such that, when the case is suspended from the supporting wall, the at least one injection device is dispensed in a horizontal orientation through the opening; and
the at least one injection device contained within the injection device storage container.

31. The system according to claim 30, wherein the at least one injection device comprises a container containing a medicament.

32. The system according to claim 30, wherein the opening is dimensioned so that only one injection device of the at least one injection device can be dispensed through the opening at a time.

33. The system according to claim 30, wherein the injection device storage container further comprises a guiding arrangement located within the case, wherein the guiding arrangement defines a path along which the at least one injection device can be guided towards the opening.

34. The system according to claim 33, wherein the guiding arrangement comprises a conduit through which the at least one injection device can move.

35. The system according to claim 33, wherein the guiding arrangement comprises a sloping lower guide surface, wherein the lower guide surface slopes upwards towards the opening in the upper panel of the case.
